(19) United States
(12) Patent Application Publication
(10) Pub. No.: US 2008/0046044 A1
(43) Pub. Date: Feb. 21, 2008

(54) Method and Apparatus to Provide Infrared Heating for an Animal

(76) Inventors: Timothy P. Jahnigen, Berkeley, CA (US); Lisa Tarver, Berkeley, CA (US)

Correspondence Address: CHALKER FLORES, LLP, 2711 LBJ FRWY, Suite 1036, DALLAS, TX, 75234, US

(21) Appl. No.: 11/764,044

(22) Filed: Jun. 15, 2007

Related U.S. Application Data
(60) Provisional application No. 60/813,963, filed on Jun. 15, 2006.

(51) Int. Cl. A61B 18/04; A61N 5/06

(52) U.S. Cl. 607/100; 606/27

(57) ABSTRACT

The present invention includes an infrared heating device and method including a first infrared heating source connected to a base and a second infrared heating source connected adjustably to the first infrared heating source to provide infrared heating to an animal.

### CROSS-REFERENCE TO RELATED APPLICATIONS

This application claims priority to U.S. Provisional Application Ser. No. 60/813,963, filed Jun. 15, 2006, the content of which is incorporated by reference herein in its entirety.

### TECHNICAL FIELD OF THE INVENTION

The present invention relates, in general, to the field of irradiating a surface with light, and in particular, to a method and apparatus for providing infrared heating to pets and animals.

### BACKGROUND OF THE INVENTION

Without limiting the scope of the invention, the background of the invention is described in connection with irradiating a body with light, as an example. An elevated body temperature is responsible for many therapeutic benefits and is useful as a treatment for many types of bodily disorders. A fever exemplifies one example of the therapeutic benefits of elevated body temperature. In response to many triggers, the body responds by elevating the body's core temperature to aid in the resolution of the problem, e.g., infection.

Common approaches of achieving localized elevated body temperatures in biological tissues include dielectric heating, microwave diathermy, ultrasonic heating and the application of hot compresses. Dielectric heating involves placing electrodes around the tissue to be treated; however, the field location within the tissues associated with electrode placement makes heating a specific region of tissue difficult. Microwave diathermy is an extension of the dielectric heating method using microwaves but disadvantages often outweigh the benefits. Ultrasonic diathermy is a commonly employed heating approach used in physical therapy to locally heat sub-surface or deep tissues to provide pain relief, however, tissue damage or unnecessary pain may also result from this treatment. Additionally, these treatments require an experienced operator and/or specialized equipment.

Typically, the application of light to the body surface or particular areas of a human or animal can relieve particular ailments. Many treatments relate to irradiating the body with light of different colors at particular areas to relieve particular ailments. Typically, an incandescent bulb that is filtered with color filters provides the light, e.g., an incandescent tungsten filament contains substantially all of the wavelengths of light. Components include color filters and heat shields to absorb a portion of the light energy to protect the color filters from deterioration. Narrow band pass filters are available; however, they are expensive.

### SUMMARY OF THE INVENTION

The present inventors recognized a longstanding need exists for a method and apparatus for increasing blood circulation and overall health of animals, while reducing stress and fatigue, while easing joint pain and stiffness. The present inventors recognized the need for a method and device to provide penetrating radiant heat to animals, pets and humans, without having to heat the air in between to provide these benefits. The present inventors recognized that an infrared heating source may be used to increase blood circulation and overall health, reduces stress and fatigue, and ease joint pain and stiffness without having to heat the environment.

The present inventors recognized that infrared emissions, particularly far infrared emission provides an effective therapy for arthritis and tissue injuries, while it promotes healing and stimulates tissues growth. The inventors recognized that this type of treatment could be used with any sort of animal or pet, including, but not limited to, a horse, a cat, a dog, a pig, a snake, a monkey, an iguana, a lizard, a turtle, a bird, a bear, a mule, a donkey, a goat, a cow, a lamb, a bull or any other animal including exotic animals.

The inventors recognized the need for an infrared heating device for relief of pain and other ailments in animals. The device includes an infrared heating source attached to a base to provide an infrared heating device for animals. Alternatively, the infrared heating device includes a first infrared heating source connected to a base and a second infrared heating source connected adjustably to the first infrared heating source to provide infrared heating to an animal.

The present invention provides an infrared heating device for animals. The infrared heating device includes a first ceramic infrared heating source connected to a base and articulately connected to a second ceramic infrared heating source. The infrared heating source emits radiation with a wavelength of between about 5 and about 1,000 μm. In one embodiment, the wavelength of between about 5 to 10 μm, and more specifically wavelength of between about 8.5 and about 9 μm. The second ceramic infrared heating source can be adjusted from between 0 degrees and 180 degrees relative to the first ceramic infrared heating source to provide infrared heating to an animal. A power supply is in electrical communication with the first ceramic infrared heating source and the second ceramic infrared heating source; and a control unit is in electrical communication with the power supply, the first ceramic infrared heating source, the second ceramic infrared heating source or combinations thereof to control the function, the temperature, the duration, the intensity, the wavelength or combination thereof.

Another example includes an infrared heating device for relief of pain and other ailments in animals. The device includes a first infrared heating source connected to a base and a second infrared heating source connected adjustably to the first infrared heating source to provide infrared heating to an animal. The first infrared heating source, the second infrared heating source or both infrared heating sources include one or more light emitting diodes positioned to emit light or a ceramic emission device.

The present invention also includes a method of infrared heat treating an animal by positioning an animal about an infrared heating device and activating the infrared heating device to provide infrared heating to the animal. The infrared heating device includes a first infrared heating source connected to a base and a second infrared heating source connected adjustably to the first infrared heating source to provide infrared heating to an animal.

### BRIEF DESCRIPTION OF THE DRAWINGS

For a more complete understanding of the features and advantages of the present invention, reference is now made to the detailed description of the invention along with the accompanying figures and in which:

FIG. 1 illustrates certain features of an infrared heating device according to an embodiment of the present invention; and

FIG. 2 illustrates certain features of an infrared heating device according to an embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The terminology used and specific embodiments discussed herein are merely illustrative of specific ways to make and use the invention and do not delimit the scope of the invention.

The present inventors recognized that infrared and more specifically far infrared emissions have the ability to penetrate, refract and radiate and therefore may be absorbed by a body because of its deep penetrating ability. For example, far infrared penetrates through the skin to the subcutaneous tissue and is transformed into heat energy with little energy being lost to heating the atmosphere. The thermal effect within the deep layers of tissues causes blood vessels in capillaries to dilate, promoting a better blood circulation and providing warmth to the body, while reducing stress and fatigue and easing joint pain and stiffness.

The inventors recognized the need for an infrared heating device for the relief of pain and other ailments in animals and pets. The device includes a first infrared heating source connected to a base and a second infrared heating source connected adjustably to the first infrared heating source to provide infrared heating to an animal. The present inventors also recognized a need for a method and device for increasing blood circulation and overall health, while reducing stress and fatigue and easing joint pain and stiffness. The present inventors recognized the need for a method and device that provides radiant heat to animals, pets and humans, without having to heat the entire room or area.

Generally, the infrared heating device can operate at temperatures of up to about 200 degrees F., while remaining safe to the touch with few or no hot surfaces or high temperature areas to burn or injure. The present invention may be used to produce any temperature between ambient temperature and 300 degrees Fahrenheit. The present invention may have a variable temperature controller that allows infinite variations within the temperature range or a selectable temperature controller that has individual settings, e.g., a high setting that corresponds to about 180 degrees Fahrenheit and a low setting that corresponds to about 140 degrees Fahrenheit. The far infrared emissions heat the skin directly, rather than warming the air first and heating the room as well as the skin. The present device also provides a portable and easy to assemble device that allows the elderly and non-mechanically inclined to benefit from the present invention without the reliance on assistance from others.

The present invention includes a base that is positioned on a surface (e.g., table, floor, chair, etc.) and has a mechanism for attaching one or more infrared heating sources. The attachment may be in the form of a peg and hole, slot and extension, bolt and screw or similar device known to the skilled artisan. In addition, the base may have attachments for multiple infrared heating sources. In some embodiments, the base includes connections for controls, timers, activators, and the infrared heating source. These connections may be made through a fitting that must be connected or an automatic connected when the device is assembled.

A first infrared heating source is enclosed or housed in an enclosure that may totally enclose the infrared heating source, e.g., protecting it from the outside. The enclosure may include a shielding device that directs the emissions in a specific direction or dissipates heat from the device. Generally, the enclosure will be rectangular in shape; however, other shapes are equally operable and include, but not limited to, an oval shape, circular shape, triangular shape, polygonal shape, free-formed shape and so forth. The dimensions of the enclosure will dictate the number, size and location of the infrared heating source. Some embodiments will have connections on or about the first infrared heating source enclosure for the connection of additional infrared heating source enclosures. The connection includes an electrical connection and a physical connection. The physical connection may be in the form of a peg and hole, slot and extension, bolt and screw or similar device known to the skilled artisan. This will allow the additional infrared heating source enclosure to be positioned to the desired location relative to the animal. Although the general configuration is to connect the infrared heating source and infrared heating source enclosure generally vertically, the configuration may encompass horizontally connected infrared heating source enclosures or both horizontal and vertical configurations to expand the coverage of the device in a modular manner.

One example includes a base that connects with an aperture and pen connection to a first infrared heating source housed in an enclosure. The first infrared heating source housed in an enclosure is then connected to a second infrared heating source housed in a second enclosure through an aperture and flexible pin to allow the second enclosure to be moved or positioned. Another example includes a base that connects with nut and bolt to a first infrared heating source housed in an enclosure. The first infrared heating source housed in an enclosure is then connected to a second infrared heating source housed in a second enclosure through a nut and bolt configuration that allows the second enclosure to be moved or positioned before securing.

For example, large animals like mastiffs or cows may use a large enclosure to allow larger infrared heating sources or more infrared heating sources to treat the animal. Alternatively, more infrared heating devices or panels may be added to the device. In addition, these devices may be incorporated into houses, floors, dog houses, barns, fences, sheds, and so forth.

The present invention provides an infrared heating device for animals, e.g., exotic animals, zoo animals, farm animals and pets. The device includes a first infrared heating source connected to a base and a second infrared heating source connected adjustably to the first infrared heating source to provide infrared heating to the animal. The base positions the first infrared heating source generally vertically, generally horizontally or at any angle between generally vertically and generally horizontally. The first infrared heating source can be positioned in any angle or position necessary and may range from 180 degrees to 0 degrees. In some instances, the base may be weighted to increase stability.

The present invention may also include a second infrared heating source housed in a second housing or enclosure. The second infrared heating source may include a removable connection to the first infrared heating source housing or enclosure. This allows the second infrared heating source to be connected as necessary. In addition, the second infrared heating source may be adjusted from between 0 degrees and 360 degrees relative to the first infrared heating source. The second infrared heating source may be connected to the first infrared heating source through a hinged joint. The hinged joint may be a single hinge, multiple hinges or articulatable members, positionable members, flexible tubing and the like. In addition, the present invention may be configured with numerous infrared heating sources positioned to allow an overlap of the emissions. For example, a first infrared heating source may be positioned substantially vertically with a second infrared heating source positioned substantially horizontally. The first source and the second source produce a region of overlap that has an increased intensity of the emission. Furthermore, the hinged joint provides ease of storage and setup, and allows portability, e.g., in an automobile, boat, plane or train.

For example, the base may be positioned on a table and the first infrared heating source positioned to extend horizontally to provide heating for an animal positioned underneath. Another example includes a base positioned on the floor and the first infrared heating source positioned to extend vertically from the base. The second infrared heating source is positioned to extend horizontally from the first infrared heating source to provide heating for an animal positioned underneath. The skilled artisan will recognize the numerous combinations and configurations available.

In addition, the present invention may include numerous controls to operate various features. For example, a simple on-off switch may be used to control the device or a timer module may be used to control the time the device is active. Other devices may be added to simplify operation, e.g., optical or mechanical sensors that activate or deactivate the device in response to the presence of an animal or pet. Other devices may include controls for temperature, duration, intensity, wavelength or combination thereof. In addition, the control may be disposed on or about the infrared heating device itself, on the cord or remotely connected either by a wireless connection and/or a wired connection. One embodiment includes an automated activation sensor to engage the device when an animal is near the infrared heating device and deactivates the device when the animal is not about the device.

The source of the infrared heating may be any source that emits radiation in the infrared wavelengths. Generally, infrared radiation is electromagnetic radiation of a wavelength longer than that of visible light, but shorter than that of microwave radiation. Infrared radiation spans three orders of magnitude and has wavelengths between about 750 nm and about 1.0 mm. In addition, a light diffuser positioned between the infrared heating source and the animal to spread the emission over a broader area.

The present invention may use sources that emit all wavelengths of infrared radiation. Infrared includes near infrared (NIR, IR-A) between about 0.75 and 1.4 μm in wavelength; short wavelength infrared (SWIR, IR-B) between about 1.4 and 3 μm; mid wavelength infrared (MWIR, IR-C); intermediate infrared (IIR), between about 3 and 8 μm; long wavelength infrared (LWIR, IR-C) between about 8 and 15 μm; and far infrared (FIR) between about 5 and 1,000 μm. Infrared sources include thermal sources, light emitting diode (LED) sources and tunable diode laser (TDL) sources.

LEDs are available in wavelengths that include 1.8, 2.8, 3.4, 3.8, 4.2, 4.8 and 5.5 microns. These are wide bandwidth sources with half power bandwidths typically 300 to 500 nm so adjacent wavelengths are also covered. Thermal sources include semiconductors, resistors, metals, alloys, filaments or combinations thereof. When in the form of a resistor, heat is applied by an electric current and may be electrically modulated at up to 10 Hz by interrupting the current flow; however, other designs may have a larger thermal mass. Common sources include ceramic far infrared elements. A metal filament may also be used as an infrared source that is enhanced and controlled by creating random surface texture. The texture may be modified to alter the reflection and absorption characteristics relative to that for a non-textured filament of the same material. Halogen tubes may also be used to provide heating by emitting radiation in the infrared wavelengths and in particular in the far infrared wavelengths. The present invention may include ceramic far infrared elements.

In addition, the present invention may combine a far infrared source with other treatments. For example, the far infrared source may be combined with thermal heaters, light therapy, physical massage and the like. For example, LEDs may be combined to produce far infrared emissions and emissions from one or more different wavelengths for relief of pain and other ailments. For example, the LEDs may be arranged in arrays and include one or more LEDs that emit wavelengths of about 950-900 nm, 989-850 nm, 849-700 nm, 699-650 nm, 649-600 nm, 599-450 nm, 449-400 nm, 399-350 nm, or 349-300 nm. More specifically, wavelengths of about 940 nm, 880 nm, 840 nm, 700 nm, 660 nm, 655 nm, 645 nm, 635 nm, 620 nm, 595 nm, 585 nm, 568 nm, 565 nm and 470 nm.

The components of the present invention may be constructed individually in-part or entirely from a variety of different materials. A non-inclusive list includes: metals, nonmetals, polymers, rubbers, alloys, plastics, carbon nanotubes, fiberglass, composites, graphite, stones, wood, carbon fiber, steel, chromolly, iron, stainless steel, brass, gold, silver, zinc, copper, nickel, titanium, aluminum or a mixture thereof. Furthermore, the present invention may be in-part or entirely coated, plated, finished, painted, varnished, covered or textured as desired. The present invention may be incorporated into a pet bed that also provides far infrared heating.

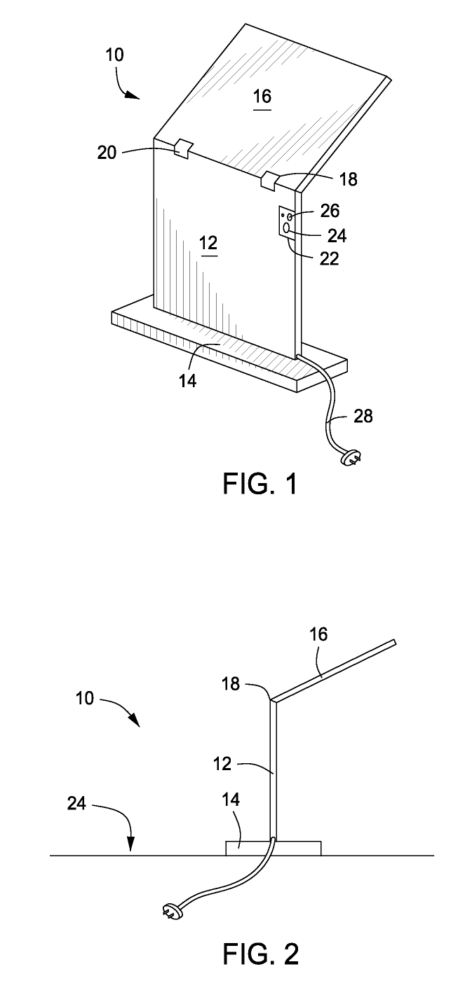

With reference to FIG. 1, the infrared heating device 10 may include a first infrared heating source 12 attached to a base 14 and a second infrared heating source 16. The first infrared heating source 12 and the second infrared heating source 16 are connected through hinges 18 and 20. Also included is control panel 22 to allow the infrared heating device 10 to be turned on and off. Other controls include a timer 24 and a temperature controller 26. The infrared heating device 10 also includes a power connection 28 to an outlet to provide electrical power for the first infrared heating source 12 and the second infrared heating source 16. Other embodiments may include pads (not shown), fans (not shown), heat lamps (not shown), electric pads (not shown), covers (not shown) or other features known to the skilled artisan.

In one example, all components are constructed from a metal frame covered by a covering that may be glass, plastic, metal, cloth, material, fiberglass, composite, or other material known to the skilled artisan. Other embodiments provide a first infrared heating source 10 that has a first ceramic far infrared element (not shown) contained within an enclosure (not shown) constructed from wood, while the second infrared heating source 16 contains a second ceramic far infrared element within a second enclosure constructed from a metal, alloys, plastic, polymer, carbon nanotubes, fiberglass, composite, graphite, stone, wood or combination thereof. This is merely for example and the skilled artisan will recognize that the components may be constructed in-part or entirely from a variety of materials known to the skilled artisan.

The first infrared heating source 12 is attached to the base 14 and second infrared heating source 16. The second infrared heating source 16 may be articulated relative to the first infrared heating source 12 to a variety of positions to allow the desired coverage, e.g., 90 degrees to 140 degrees. Generally, the second infrared heating source 16 may be articulated between about 10 degrees and 180 degrees relative to the first infrared heating source 12; however, common angles include between about 70 and 140 degrees relative to the base 14. The first infrared heating source 12 and second infrared heating source 16 include numerous articulation positions through hinges 18 and 20 or other devices known to the skilled artisan (not shown). Hinges 18 and 20 are used to secure the first infrared heating source 12 to the second infrared heating source 16 and maintain the desired angle.

FIG. 2 is a side view of the infrared heating device 10 in operation. The infrared heating device 10 is placed in contact with the floor 24. A first infrared heating source 12 is attached the base 14. The first infrared heating source 12 may be articulated to a variety of positions to allow the desired coverage. Generally, the first infrared heating source 12 may be articulated between about 10 degrees and 180 degrees relative to the base 14; however, common angles include between about 70 and 90 degrees relative to the base 14. The first infrared heating source 12 includes hinge 18 to connect to second infrared heating source 16. An articulation fastener (not shown) may also used to secure the first infrared heating source 12 to the second infrared heating source 16 and/or base 14 and maintain the desired angles.

The infrared heating device may include a first infrared heating source, a base and a second infrared heating source connected using screws, glues, epoxy, fitting frictionally, a pin locking mechanism or other mechanism known to the skilled artisan. Alternatively, the infrared heating device may be of a unibody construction. The skilled artisan will recognize that the first infrared heating source 12, the base 14 and the second infrared heating source 16 may be of different lengths, shapes, profiles and thicknesses depending on the requirements of the particular application. Additionally, the cross sectional shape of the individual components may differ depending on the application. In another example, the first infrared heating source 12, the base 14 and the second infrared heating source 16 will be covered with a variety of finishes, materials, fabrics or designs.

The connectors or hinges used in the present invention include clips, pins, screws, clamps, nails, glues, tapes, Velcro, loop and hook fasteners, slip pins, self-locking pins, fasteners, lock and key arrangements, slot and groove arrangement, and other fasteners known to the skilled artisan. When connectors or hinges are in the form of a pin, each pin may be self-activating to secure the components. For example, the components of the present invention may be assembled using a cotter pin design to secure the components using minimal effort and tools.

In addition, the present invention provides a surface mounted version of the present invention. For example, the infrared heating device 10 may be mounted on the surface including the wall or ceiling, a support beam or column, a door or other structural component. The infrared heating device 10 may be mounted using screws and brackets to attach the infrared heating device 10 to the surface. In addition, infrared heating device 10 may be removably mounted to the surface using screws, hangers, suction cups, wires or other mechanisms known to the skilled artisan. The infrared heating device 10 may include a first infrared heating source 12 attached to a first surface (e.g., a wall) and a second infrared heating source 16 may be attached to one or more brackets that extend to position the second infrared heating source 16 over the subject. The first infrared heating source 12 may attach to a first surface directly or via an adaptor or bracket. The brackets may be fixed in position or be articulatable to allow the movement of the first infrared heating source 12 and/or the second infrared heating source 16 to the desired position. The first infrared heating source 12 and second infrared heating source 16 may be articulated relative to each other to provide a variety of positions to allow the desired coverage, e.g., 90 degrees to 140 degrees. Generally, the second infrared heating source 16 may be articulated between about 10 degrees and 180 degrees relative to the first infrared heating source 12; however, common angles include between about 70 and 120 degrees relative to the base 14. The first infrared heating source 12 and second infrared heating source 16 include numerous articulation positions through hinges 18 and 20 or other devices known to the skilled artisan (not shown). In another example, the first infrared heating source 12 and the second infrared heating source 16 are connected through hinges 18 and 20. Also included is control panel 22 to allow the infrared heating device 10 to be turned on and off. The control panel 22 may be connected directly or via a wired or wireless connection. Other controls include a timer 24 and a temperature controller 26. The infrared heating device 10 also includes a power connection 28 to an outlet or power supply to provide electrical power for the first infrared heating source 12 and/or the second infrared heating source 16. The first infrared heating source 12 and the second infrared heating source 16 may be powered by the same power supply or separate power supplies. In addition, a computer may be connected to the infrared heating device 10 and used to control the various functions and accessories, e.g., heaters, fans etc.

A portable version is also included in the present invention for use where attachment to structural components is impractical or impossible. For example, the infrared heating device 10 may be attached to a portable stand that allows the positioning of the infrared heating device 10 at the desires height for the animal, while providing a stable base. One such device is known to the skilled artisan in the form of an instrument stand or IV stand, which can be fitted with the infrared heating device 10 of the present invention. In some instances, multiple stands can be used to support larger infrared heating devices or increase stability. The infrared heating device 10 may be mounted using screws and brackets to attach the infrared heating device 10 to the portable stand. In addition, the infrared heating device 10 may be removably mounted to the portable stand using screws, hangers, suction cups, wires or other mechanisms known to the skilled artisan.

The infrared heating device 10 may include a first infrared heating source 12 attached to a portable stand and a second infrared heating source 16 may be attached to one or more brackets that extend to position the second infrared heating source 16 over the subject. The first infrared heating source 12 may attach to a portable stand directly or via an adaptor or bracket. The brackets and/or the portable stand may be fixed in position or be articulatable to allow the movement of the first infrared heating source 12 and/or the second infrared heating source 16 to the desired position. In another example, the first infrared heating source 12 and the second infrared heating source 16 are connected through hinges 18 and 20. Also included is control panel 22 to allow the infrared heating device 10 to be turned on and off. The control panel 22 may be connected directly or via a wired or wireless connection. Other controls include a timer 24 and a temperature controller 26. The infrared heating device 10 also includes a power connection 28 to an outlet or power supply to provide electrical power for the first infrared heating source 12 and/or the second infrared heating source 16. The first infrared heating source 12 and the second infrared heating source 16 may be powered by the same power supply or separate power supplies. In addition, a computer may be connected to the infrared heating device 10 and used to control the various functions and accessories, e.g., heaters, fans etc.

The infrared heating device may be mounted on a cart or stand with rollers or wheels to allow easy of mobility. The infrared heating device 10 of the present invention can be incorporated into a transport device such as a trailer. The infrared heating device 10 may be mounted on the inner surface of the enclosure to allow therapy during transport or provide a mobile therapy vehicle. The infrared heating device 10 may be mounted using screws and brackets to attach the infrared heating device 10 to the surface. In addition, infrared heating device 10 may be removably mounted to the surface using screws, hangers, suction cups, wires or other mechanisms known to the skilled artisan. The infrared heating device 10 may include a first infrared heating source 12 attached to a first surface (e.g., a wall) and a second infrared heating source 16 may be attached to one or more brackets that extend to position the second infrared heating source 16 over the subject. The first infrared heating source 12 may attach to a first surface directly or via an adaptor or bracket. The brackets may be fixed in position or be articulatable to allow the movement of the first infrared heating source 12 and/or the second infrared heating source 16 to the desired position. In another example, the first infrared heating source 12 and the second infrared heating source 16 are connected through hinges 18 and 20. Also included is control panel 22 to allow the infrared heating device 10 to be turned on and off. The control panel 22 may be connected directly or via a wired or wireless connection. Other controls include a timer 24 and a temperature controller 26. The infrared heating device 10 also includes a power connection 28 to an outlet or power supply to provide electrical power for the first infrared heating source 12 and/or the second infrared heating source 16. The first infrared heating source 12 and the second infrared heating source 16 may be powered by the same power supply or separate power supplies. In addition, a computer may be connected to the infrared heating device 10 and used to control the various functions and accessories, e.g., heaters, fans etc.

The components are constructed from a metal frame covered by a covering that may be glass, plastic, metal, cloth, material, fiberglass, composite, or other material known to the skilled artisan. Other embodiments provide a first infrared heating source 10 that has a first ceramic far infrared element (not shown) contained within an enclosure (not shown) constructed from wood, while the second infrared heating source 16 contains a second ceramic far infrared element within a second enclosure constructed from a metal, alloys, plastic, polymer, carbon nanotubes, fiberglass, composite, graphite, stone, wood or combination thereof. This is merely for example and the skilled artisan will recognize that the components may be constructed in-part or entirely from a variety of materials known to the skilled artisan.

Another embodiment of the present invention includes an infrared heating device 10 that is fixable to a table, e.g., surgery or treatment table. The infrared heating device 10 may be connected with clamps, inserts and other fasteners known to the skilled artisan. The infrared heating device 10 may be used to achieve normothermia in a veterinary post surgical situation. In addition, the infrared heating device 10 may be used to maintain normothermia during veterinary surgery, treatment and grooming.

Another embodiment includes the use of the infrared heating device 10 to alter or modulate (e.g., increase or decrease) metabolism and alkalizes acids in humans, and a direct correlation can be seen when observing wildlife behavior in nature, e.g., Lions, cats and dogs basking in the sun after eating. The infrared heating device 10 of the present invention may be used to alter, modulate, improve and/or optimize the digestion and assimilation of a high protein diet. The metabolic energy required to process high protein is enhanced in nature by animals instinctively laying in the sun. This cannot be achieved with convective and or conductive heat, which are commonly found in the modern home.

The present invention includes an infrared heating device 10 that uses focusable panels to adapt, concentrate and/or focus the intensities of the emissions. The first infrared heating source 12 and the second infrared heating source 16 may include a panel that may be distorted (e.g., curved or bent) in some manner to adapt, concentrate and/or focus the intensities of the emissions to produce the desired effect.

The present invention includes a multiple panel infrared heating enclosure (e.g., trailer, doghouse, kennel, cage, tent, cave, zoo enclosure, transport container (e.g., trucks, cars, trains, planes and boats) and other enclosures. The infrared heating device 10 may include infrared heating sources on one or more walls or portions of the walls, the ceiling or portions of the ceiling or combinations thereof. The infrared heating device may also be connected to a control panel to allow the infrared heating device 10 to be turned on and off, and other controls include a timer 24 and a temperature controller 26. The control panel 22 may be connected directly or via a wired or wireless connection. The infrared heating device 10 also includes a power connection 28 to an outlet or power supply to provide electrical power for the first infrared heating source 12 and/or the second infrared heating source 16. The first infrared heating source 12 and the second infrared heating source 16 may be powered by the same power supply or separate power supplies. In addition, a computer may be connected to the infrared heating device 10 and used to control the various functions and accessories, e.g., heaters, fans etc.

In addition to the multiple panel infrared heating device, the present invention also includes a single panel infrared heating device 10 of a variety of different sizes to accommodate specific applications. In some applications, the present invention may be incorporated into the floor or a sub-floor of an enclosure. The present invention is configured specifically around animal physiology and behavior allowing positioning of the device where animals can use them instinctively.

In addition, the present invention may also include alternate sources of power for the infrared heating device to allow operation during power outages or in locations without power such as fields and farms. Such power sources include battery power, power converters to convert a 12 volt source to usable power solar power, capacitors and so forth.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims. The apparatus, method and/or kit disclosed and claimed herein can be made and executed without undue experimentation in light of the present disclosure. While the apparatus, method and kit of this invention have been described in terms of preferred embodiments, it will be apparent to those of skill in the art that variations can be applied to the apparatus and kit and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An infrared heating device for animals comprising:
a first infrared heating source that emits radiation with a wavelength of between about 7 and about 10 μm connected adjustably to a second infrared heating source that emits radiation with a wavelength of between about 7 and about 10 μm and positioned to provide infrared heating to an animal.

2. The device of claim 1, wherein the first infrared heating source is free standing or mounted to a base, a wall, a post, a stand, a table, a ceiling, a floor, a door, a cart, or an enclosure.

3. The device of claim 1, wherein the first infrared heating source, the second infrared heating source or both infrared heating sources comprise a ceramic far infrared element.

4. The device of claim 1, wherein the first infrared heating source, the second infrared heating source or both infrared heating sources comprise one or more light emitting diodes positioned to emit light.

5. The device of claim 1, wherein the first infrared heating source and the second infrared heating source may be adjusted from between 0 degrees and 180 degrees relative to the each other.

6. The device of claim 1, further comprising a third infrared heating source that emits radiation with a wavelength of between about 7 and about 10 μm in communication with the first infrared heating source and the second infrared heating source and positioned to provide infrared heating to an animal.

7. The device of claim 1, further comprising a control in communication with the infrared heating device to control the operation, the temperature, the duration, the intensity, the wavelength or combination thereof.

8. The device of claim 1, wherein the infrared heating device comprises an automated activation sensor to engage the device when the animal is about the infrared heating device and deactivate the device when the animal is not about the device.

9. A infrared heating device for relief of pain and other ailments in animals comprising:
one or more infrared heating sources that emit radiation at a wavelength of between about 7 and about 10 μm to provide infrared heating to an animal.

10. The device of claim 9, wherein the one or more infrared heating sources each comprise a ceramic far infrared element.

11. The device of claim 9, wherein the one or more infrared heating sources comprise a first infrared heating source positioned between 0 degrees and 180 degrees relative to a second infrared heating.

12. The device of claim 11, wherein the first infrared heating source and the second infrared heating sources are connected through one or more articulatable joints.

13. The device of claim 9, further comprising one or more light emitting diodes positioned to emit light about the animal for relief of pain and other ailments.

14. The device of claim 13, further comprising a light diffuser positioned between the light emitting diodes and the animal to spread the emitted light over a broader path.

15. The device of claim 13, wherein the one or more light emitting diodes emit wavelengths of about 950-900 nm, 989-850 nm, 849-700 nm, 699-650 nm, 649-600 nm, 599-450 nm, 449-400 nm, 399-350 nm, or 349-300 nm.

16. The device of claim 13, wherein the one or more light emitting diodes are arranged in arrays.

17. A method of treating an animal using infrared heat comprising the steps:
positioning an infrared heating device for use by an animal, wherein the infrared heating device comprises a first infrared heating source that emits radiation with a wavelength of between about 7 and about 10 μm connected adjustably to a second infrared heating source that emits radiation with a wavelength of between about 7 and about 10 μm to provide infrared heating to an animal; and
activating the infrared heating device to provide infrared heating to the animal.

18. The method of claim 17, wherein the first infrared heating source, the second infrared heating source or both infrared heating sources comprise one or more ceramic far infrared elements.

19. The method of claim 17, further comprising one or more light emitting diodes positioned to emit light.

20. The method of claim 17, wherein the second infrared heating source may be adjusted from between 0 degrees and 180 degrees relative to the first infrared heating source.

21. An infrared heating device for animals comprising:
a first ceramic infrared heating source which emits radiation with a wavelength of between about 7 and about 10 μm positioned from between 0 degrees and 180 degrees relative to a second ceramic infrared heating source that emits radiation with a wavelength of between about 7 and about 10 μm to provide infrared heating to an animal;
a power supply in communication with the first ceramic infrared heating source and the second ceramic infrared heating source; and
a control unit in communication with the power supply, the first ceramic infrared heating source, the second ceramic infrared heating source or combinations thereof to control the function, the temperature, the duration, the intensity, the wavelength or combination thereof.
